(19) United States
(12) Patent Application Publication
(10) Pub. No.: US 2004/0091460 A1
(43) Pub. Date: May 13, 2004

(54) Preparation of spheroids

(76) Inventors: Wendy Purcell, Bristol (GB); Jinsheng Xu, Bristol (GB)

Correspondence Address: SPENCER, FANE, BRITT & BROWNE, 1000 WALNUT STREET, SUITE 1400, KANSAS CITY, MO, 64106-2140, US

(21) Appl. No.: 10/433,928

(22) PCT Filed: Dec. 7, 2001
(86) PCT No.: PCT/GB01/05445

(30) Foreign Application Priority Data
Dec. 7, 2000 (GB) 0029847.1

(51) Int. Cl. A61K 45/00; B29B 9/00

(52) U.S. Cl. 424/93.7; 264/5

(57) ABSTRACT

There is disclosed a method for the formation of spheroids comprising: (a) obtaining and/or deriving a plurality of cells from a tissue or an organ or from a part thereof; (b) orbitaly rotating the cells in a fluid medium at a first speed and for a time sufficient for spheroids to form; and (c) further orbitally rotating the spheroids formed in step (b) in a fluid medium at a second speed or speeds, slower than said first speed.

The present invention relates to artificially produced aggregates of cells in the form of three dimensional structures which are known as spheroids, reaggregates, organoids, and cryoids.

Spheroids have significant potential to be used as in vitro models of different tissue types, and in particular have the potential to be used as models for testing novel chemical entities (NCE's) and/or new therapeutic agents.

WO-A-98/35021 describes methods for the formation of spheroids which are then cryopreserved. In one method of forming the spheroids, a period of orbital rotation at a first speed is followed by a period of orbital rotation at a second speed which is higher than the first speed.

Prior art methods for the preparation of spheroids tend to result in populations of spheroids which are not uniformly sized. This is an undesirable characteristic from at least three points of view. Firstly, differently sized spheroids tend to be viable for different periods of time. Hence, a population of spheroids made in a given batch will include spheroids which have relatively shorter and relatively longer viability. This is undesirable in a material which is to be used as a tissue model to evaluate drug candidates and/or NCE's. Secondly, differently sized spheroids may respond differently to drug candidates and/or NCE's e.g. in terms of the extent to which a drug candidate and/or NCE will penetrate into the spheroid. This may make analysis of the results difficult. Thirdly, heterogeneous-sized spheroids will display variable functional responses following exposure to drugs and this confounds data analyses, including assessment of efficacy and/or toxicity.

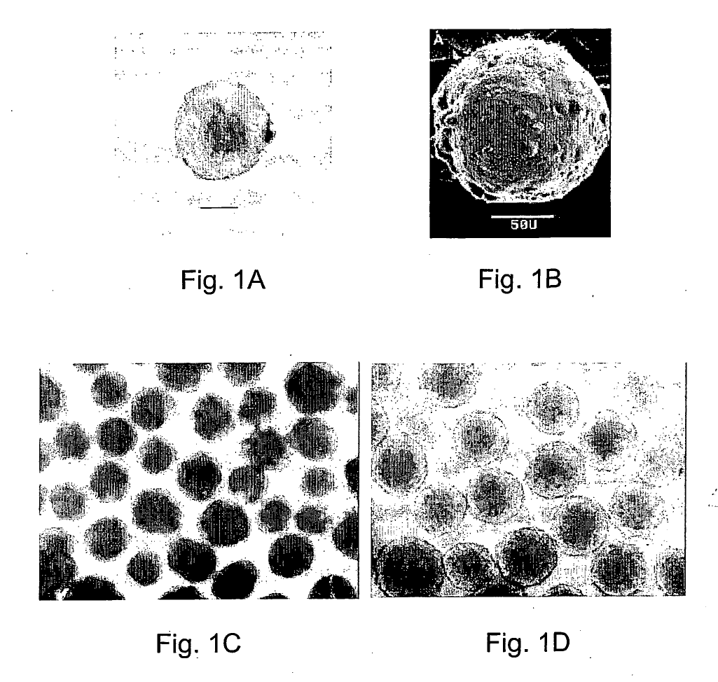

It would be desirable to be able to provide a method for making spheroids in which the resulting spheroid population is uniform in size (See FIG. 1).

According to a first aspect of the invention, there is provided a method for the formation of spheroids comprising:

(a) obtaining and/or deriving a plurality of cells from a tissue or an organ or from a part thereof;

(b) orbitally rotating the cells in a fluid medium at a first speed and for a time sufficient for spheroids to form; and

(c) further orbitally rotating the spheroids formed in step (b) in a fluid medium at a second speed or speeds, slower than said first speed.

Spheroids made in accordance with the present invention can be used as in vitro models and are therefore advantageous alternatives to using live animals. Preferred models are mammalian (e.g. human, non-human primate, porcine, rodent) or avian (e.g. chick) models.

As used herein, a spheroid means a three-dimensional structure, normally spherical in shape, which does not occur in nature and which consists of a reaggregate of cells—typically containing 10³ or more cells—of a tissue or of an organ or formed from cell lines either alone or in combination.

An advantage of the method of the present invention is that it enables the production of a uniform population of spheroids. Thus, the method of the invention may be conducted such that a substantial portion (for example at least 75%, more preferably at least 90%) of the spheroids formed in the method have a size which lies within a relatively narrow size range and, morphologically, the shape of an individual spheroid is regular and has a smooth surface.

A suitable narrow size range, measured in terms of the diameter of a spheroid, is from 100 μm to 350 μm, the upper limit of this range being a practical upper limit which arises in use because, at larger diameters, and depending on the tissue type, the centre of the spheroid undergoes necrosis due to inefficient diffusion rates of nutrients into and waste products out of the spheroid. Typically, therefore, the method of the invention may be employed to make a population of spheroids in which at least 75%, preferably at least 90%, of the spheroids have a diameter which lies within the range of from 100 to 350 μm.

A more preferred narrow size range, which is appropriate for spheroids made from most tissue types, is from 150 to 200 μm in diameter. The method of the invention is preferably employed to make a population of spheroids in which at least 75%, preferably at least 90%, of the spheroids have a diameter which lies within this range.

The spheroids made in accordance with the invention may be made up of one or more different cell types. If a plurality of different cell types are present they may form different layers of the spheroid.

The term “tissue” as used herein is taken to mean an organised selection of cells having a common function. The term “organ” is taken to mean an organised collection of “tissues” having a common function. The term “cell line” is taken to mean a continuous cell culture derived from cells subject to transformation or otherwise having acquired the ability to divide continuously.

The “tissue” or “organ” need not be completely intact to be used in the present invention since parts of whole tissues or organs (which may be obtained via biopsies) can be disrupted to individual cells/small groups of cells before being re-aggregated to form spheroids in accordance with the invention.

Cells for use in producing spheroids in accordance with the invention can be derived from any suitable tissue source, including infected tissue. For spheroids comprising neuronal cells (e.g. brain spheroids), foetal tissue is preferred. For spheroids comprising other cells, tissue from both embryonic/foetal and non-foetal (e.g. adult sources) can generally be used. Liver cells are particularly useful since they can be used to produce spheroids which retain some of the characteristics of the liver e.g. albumin secretion, urea secretion, glucose secretion and can therefore be used to model in vitro the metabolism of substances in the liver and to explore general cytotoxic and specific hepatotoxic effects. This is useful, for example, in determining whether or not particular substances are likely to be toxic following metabolism by the liver and/or are directly toxic to the liver cells (i.e. hepatotoxins) or interfere with generic cellular functionality.

Spheroids can in principle be produced from any desired tissue or organ from any animal by disrupting a sample of the tissue or organ, preferably to individual cells or to small groups of cells. For example, mechanical disruption such as via gentle trituration through a Pasteur pipette can be employed for retinal and brain tissues. Alternatively, enzymatic digestion methods can be used, for example for liver cell dissociation.

Cells from cell lines may also be used. These may be initially cultured as a monolayer to generate more cells; trypsinization may be used for cell dissociation of a monolayer cell culture.

It is preferred that the cell sample employed in making the spheroids in accordance with the invention is one which has a high cell viability, preferably greater than or equal to 75% viability.

In the method of the invention, the cell suspension is first subjected to an orbital-rotation at a first speed followed by a period of orbital rotation at a second speed and/or speeds, slower than the first. The actual values of the speeds of rotation will vary among cell types, but typically the first speed of rotation will be greater than 77 rpm, and the second speed of rotation will be less than 80 rpm. Thus, for instance the first speed of rotation may be from 77 to 90 rpm, preferably 83-87 rpm, and the second speed of rotation may be from 77 to 90 rpm, preferably 83-87 rpm.

The duration of rotation for each of the first and second periods of orbital rotation may be 1 to 4 days at the first speed, preferably 1 to 2 days at the first speed, and maintaining the second and/or subsequent lower speeds for the remainder of the culture period, e.g. 2 to 45 days, or preferably 2 to 35 days.

The initial cell seeding density of the cell suspension for orbital rotation will vary from cell type to cell type, but typically may range from 3×10⁵ to 5×10⁶ cells per ml.

In one embodiment of the invention, orbital rotation is carried out on a small volume of medium containing cells, for example from 2.5-4.5 ml in a single- well. This volume normally contains enough nutrients for cells at proper density for up to two days, dependent upon the cell type and its metabolic demands, and makes shaking more efficient. A plate containing a plurality of such wells, for example 6 wells, may be used.

The fluid medium used to suspend the cells during the orbital rotation steps may comprise any one or more of the following components: sodium chloride, glucose, serum, antibiotics, defined media supplements. The medium used would vary according to the cell type. However, any suitable standard media may be used. A typical culture medium would include a balanced salt solution and glucose and the pH would need to be monitored and controlled.

Spheroids produced by the present invention may be cryopreserved as described in WO98/35021.

Thus in a cryopreservation step, the spheroids may be preserved with a cryopreservative.

Various cryopreservants can be used, although DMSO is preferred.

Other cryopreservants include permeating cryoprotectants besides DMSO can however be used: e.g. glycerol, 1,2-propanediol, acetamide, ethylclycol and propylene glycol (Karlsson J O M, & Tomer M. Long-term storage of tissues by cryopreservation: critical issues. Biomaterials 1996: 17:243-256; Chao N H, Chiang C P, Hsu H W, Tsai C T and Lin T T (1994). Toxicity tolerance of oyster embryos to selected cryoprotectants. Aquatic Living resource, 7(2):99-104). Non-permeating cryopreservants may even be used: e.g. methylcellulose polyvinyl pyrollidone, hydroxyethyl starch and various sugar-based cryopreservants.

Desirably the cryopreservant is at a level of at least 5% v/v (e.g. from 10-20% v/v) with respect to a composition comprising the spheroids and the cryopreservant immediately prior to cryopreservation.

It is believed that the best results can be achieved if the spheroids are cooled in a step-wise manner. Therefore in preferred methods of the present invention spheroids are cooled and then maintained within a given temperature range before being cooled further. Without being bound by theory, it is possible that this procedure may give the cells constituting a spheroid sufficient time to acclimatize to cold temperatures and thereby to avoid cell death or damage which might otherwise occur.

The spheroids can be cooled to a temperate of from 1 to 8° C. (e.g. from 2 to 6° C. and preferably of about 4° C.) and held for a period before further cooling. This period may be a period of at least 10 mins, preferably a period of at least 30 mins and typically a period of 40-60 mins. Such cooling can be conveniently achieved by using a laboratory refrigerator.

The spheroids may then be cooled to a temperature of from 0 to −50° C. (e.g. from −10 to −30° C., preferably of from −15 to −25° C.) and held for a period before further cooling. This can be conveniently achieved by using a laboratory freezer. This period may be a period of at least 30 mins and is preferably of at least 1 hour (e.g. 1-6 hours).

The final step in the cooling procedure will usually be a rapid cooling to a temperature of below −100° C. e.g. below −190° C. This can be done using liquid nitrogen as a coolant, into which is placed a resilient container containing the spheroids and the cryopreservant.

Following cryopreservation the spheroids are thawed. This can be done by placing containers containing the spheroids in a water bath at 37° C., typically for at least 2-3 mins. Once thawed, the cryopreservation medium can be removed e.g. by centrifugation. The spheroids may then be washed and further traces of the cryopreservation medium removed (e.g. by centrifugation).

It is desirable that orbital rotation is also performed after thawing of the spheroids. This can be done at one or more rotation speeds of at least 50 rpm (e.g. of from 50 to 77 rpm). If two or more different orbital rotation speeds are used then a period of rotation at a first speed will usually be followed by, a period of rotation at a second speed which is higher than the first speed.

Preferably the spheroids are rotated (post-cryopreservation) at a rotation speed of from 50 to 70 rpm (e.g. of about 60 rpm). Typically this may be done for at least 6 hours (e.g. 12-48 hours). This may be followed by a period of rotation at a higher speed (e.g. of at least 70 rpm), preferably of about 75 rpm. This may typically be for a period of at least 7-10 days or longer.

The thawed spheroids can generally be maintained in cultures post-cryopreservation for a period of several days.

Embodiments of the invention will now be described, by way of example only, and with reference to the following figures:

FIG. 1 shows a uniform spheroid population made according to the present invention;

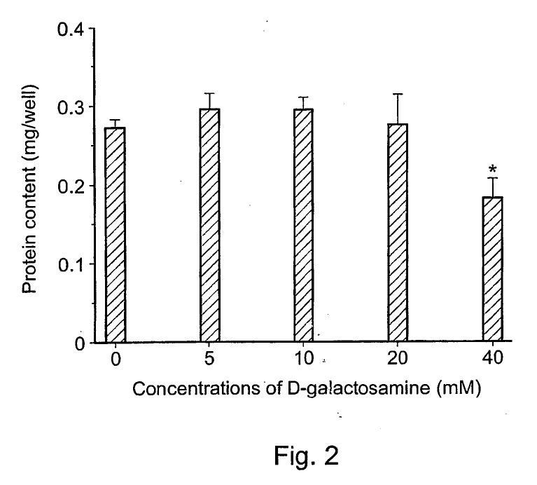

FIG. 2 is a graph showing the effect of varying concentrations of D-galactosamine on protein content in rat liver spheroids 24 hours after exposure;

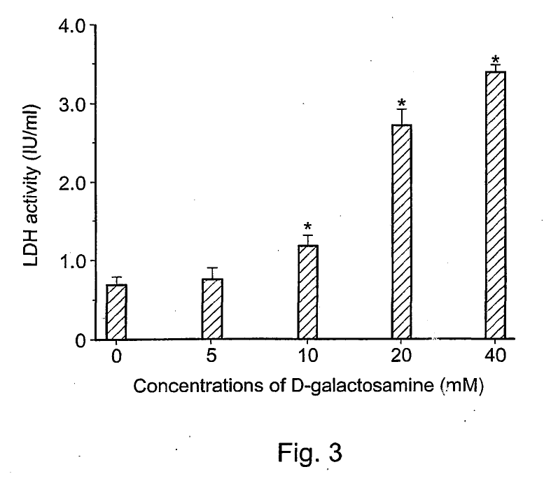

FIG. 3 is a graph showing the effect of varying concentrations of D-galactosamine on LDH leakage of hepatocyte liver cells 24 hours after exposure;

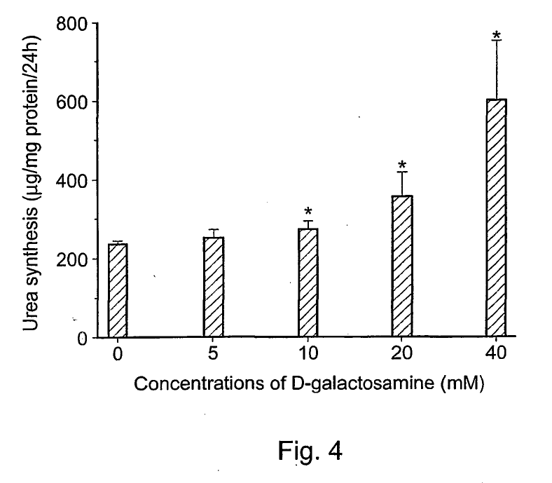

FIG. 4 is a graph showing the effect of varying concentrations of D-galactosamine on urea synthesis by rat liver spheroids;

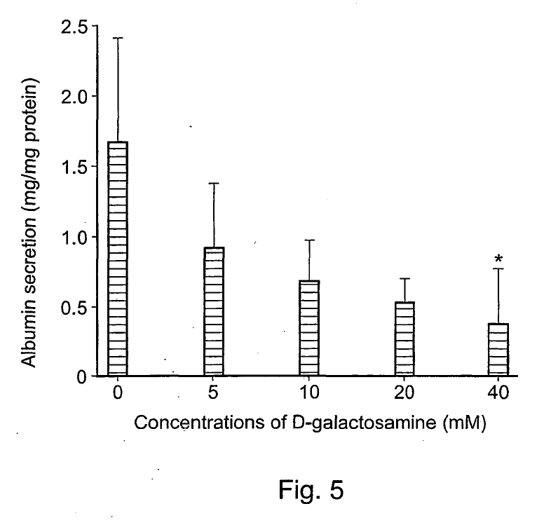

FIG. 5 is a graph showing the effect of varying concentrations of D-galactosamine on albumin synthesis by rat liver spheroids; and

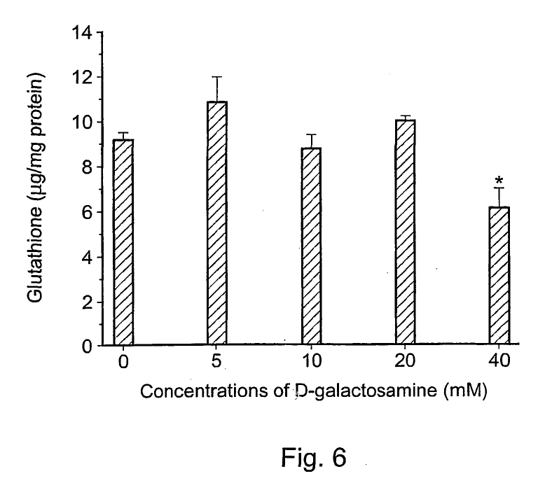

FIG. 6 is a graph showing the effect of varying concentrations of D-galactosamine on glutathioine concentrations in rat liver spheroids; and

In the case of the data shown in FIGS. 2 to 6, the results are the mean ±SEM, n=6.*:p<0.05.

### EXAMPLE

In this example, adult rat liver spheroids are made according to the invention:

### Hepatocyte Spheroid Culture

Liver spheroids were prepared from the liver of male Wistar rats by a two-step collagenase perfusion method described by Seglen P. O. ((1976). Preparation of isolated rat liver cells. Methods Cell. Biol. 13, 29-38) and modified by Lazar, A.; Peshwa, M. V., Wu, F. J., Chi, C. M., Cerra, F. B., and Hu, W. S. (1995). Extended liver-specific functions of procirne hepatocyte spheroids entrapped in collagen gel. In Vitro Cell Biol Anim. 31, 340-346). Viability of the isolated liver cells was determined by tyrpan blue dye exclusion i.e. an aliquot of isolated liver cells was mixed with an equal volume of trypan blue dye (1.0% w/v in isotonic saline) and incubated at room temperature for a minimum period of 5 minutes. Only isolated liver cell preparations with viability above 80% were used to prepare liver spheroids. The cell suspension was diluted with culture medium (hepatocyte medium supplemented with 10% FCS, 200 mM L-glutamine, 2 ng/ml insulin, 100 U/ml penicillin and 100 μg/ml streptomycin sulfate) to give a cell density of 5×10⁵ cells/ml. The diluted cell suspension was dispersed into 6-well plates, 3 ml/well. The plates were incubated at 37° C., in a 5% CO₂ incubator on a gyrotatory shaker (New Brunswick) at an initial rotation speed of 85 rpm for the first 24 hr and 77 rpm thereafter. The plates were rotated at this speed for the duration of the study (up to 45 days, but typically 2-10 days). 1.5 ml of old medium was replaced with 2.0 ml fresh medium for each well every other day. Media exchange was carried out over the duration of the study (up to 45 days, but typically 2-10 days).

The spheroids made are shown in FIG. 71 and have a uniform size typically 170 μm, of which >80% were in the range of 160-180 μm.

The spheroids made in this example from adult rat liver spheroid cultures were then tested to investigate their suitability as an in vitro model for the investigation of the effects of a model hepatoxin, as follows:

### Effects of the Repatoxin, D-Galactosamine, On In Vitro Rat Liver Spheroids

D-Galactosamine (GalN) normally exists in vivo in an acetylated form in certain structural polysaccharides and is a well-known hepatotoxic agent which can produce acute liver necrosis. Therefore, it has been widely used as a model hepatotoxin in studies of liver damage. GalN in liver is converted to uridine diphosphate galactosamine and causes rapid and extensive uridine triphosphate (UTP) depletion and a decrease in ATP, that subsequently cause secondary biochemical changes in hepatocytes including the inhibition of RNA and protein syntheses.

### Chemicals and Medium

L-Glutamine was obtained from GibcoBril. Foetal calf serum (FCS) was purchased from Imperial. Hepatocyte medium, D-galactosamine (GalN), penicillin and streptomycin sulfate and other chemicals and reagents were obtained from Sigma.

### Statistics

Data from all biochemical assays were analysed by ANOVA and two-sample students t-test. P<0.05 was accepted as significant for both tests.

### Intoxification for Spheroid Functional Tests

Procedures: The old hepatocyte spheroid media in the 6-well plates was totally replaced by 3 ml of either 0, 5, 10, 20 or 40 mM GalN solution in serum-free hepatocyte medium (supplemented with 200 nM L-glutamine, 2 ng/ml insulin, 100 U/ml penicillin and 100 μg/ml streptomycin sulfate), one plate for each concentration. The plates were cultured on a gyrotatory shaker at 75 rpm in an incubator at 37° C. in CO₂/O₂ 5/95% and water saturation environment for 24 hr.

### Sample Preparation

Spheroids in each well of 6-well plates were collected 24 hours after exposure to GalN and transferred to an ependorff tube. The tubes were centrifuged at 900 g for 3 min and the supernatant retained for urea, albumin and lactate dehydrogenase (LDH) assays. The spheroids were then homogenized in 1 ml of homogenizing buffer (containing NaH₂PO₄2H₂O,, 2 mM; Na₂HPO₄, 2 mM; EDTA, 0.5 mM; and NaCl, 145 mM). Each homogenate of 100 μl of 10% TCA, vortexed and used for the glutathione assay. The remaining homogenates were used for protein assay. All samples were stored at −20° C. until assay.

### Biochemical Assays

1. Protein

Spheroids were homogenized in homogenisation buffer containing NaH₂PO₄2H₂O, 2 mM; Na₂HPO₄, 2 mM; EDTA, 0.5 mM; and NaCl, 145 mM. Total protein was assessed according to the method developed by Bradford (1976). Homogenates were diluted, 1:2, and 10 μl was added to each well of a 96-well plate. The protein content was determined by Micro Protein Kit (Sigma, Cat. No. 610-A). Micro protein reagent was diluted 1:5 with distilled water. The diluted protein reagent of 250 μl was added to each well. The plate was allowed to stand for 5 min at room temperature. Absorbance was read on a microplate reader (Multiscan RC, Labsystem) at 595 nm.

Protein content in liver spheroids are shown in FIG. 2. The total protein content in 20 mM or lower GalN groups was not affected. However, GalN at 40 mM significantly decreased the total protein content of spheroids compared with the control (p<0.05). Some spheroids were also damaged at this highest concentration, indicating that this concentration of GalN caused acute cell death.

2. Albumin

Albumin concentration in the medium was determined by Albumin Reagent Kit (BCG, Sigma). The original reagent was diluted 1:6 with distilled water. The method was modified to be suitable for a 96-well plate format. A 20 μl aliquot of media was added to each well and followed by 200 μl of diluted reagent. Absorbance was read on a microplate reader (Multiscan RC, Labsystems) at 630 nm.

FIG. 5 shows albumin synthesis of liver spheroids after exposure to GalN. Albumin synthesis after exposure to GalN showed a concentration-dependent decrease. The decrease was significant at 40 mM GalN compared with the control (p<0.05).

3. Urea

Urea concentration in the media was determined using a Urea Nitrogen Kit (Cat. 640-A, Sigma). The method was modified in order to be suitable for a 96-well plate format and assays using a microplate reader. Samples were diluted 1:2 with PBS. Total ammonium nitrogen of each sample was determined by adding 25 μl urease solution to each well followed by 10 μl of diluted medium. The plate was shaken to mix and the reaction allowed to develop at room temperature for 20 min. Then, the following reagents were added to each well in order: 50 μl phenol nitroprusside sodium, 50 μl alkaline hypochlorite solution, and 200 μl distilled water. The plate was shaken to mix and kept at room temperature for 30 min. A blue colour developed and the absorbance was read on a microplate reader (Multiscan RC, Labsystems) at 590 nm. Free non-urea nitrogen was determined by adding 25 μl PBS without urease followed by 10 μl medium. The remaining assay steps and reagents were as above for the total ammonium nitrogen assay. The urea concentration of each sample was obtained by subtracting free non-urea nitrogen from total ammonium nitrogen.

After deduction of free non-urea ammonia, urea synthesis of liver spheroids showed a concentration-dependent increase 24 hr after exposure to GalN. The synthetic rates of urea in the spheroids at 10 mM or higher concentrations of GalN were significantly higher than that in control (all p<0.05), see FIG. 4.

4. Gluthathione (GSH)

Glutathione levels in spheroids were determined by the fluorimetric method (Hissin P. J., and Hilf (1976). A fluorimetric method for the determination of oxidised and reduced glutathione in tissues. Anal. Biochem. 74, 214-226). A 100 μl aliquot of spheroid homogenate from each sample was mixed with an equal volume of, 10% trichloroacetic acid (TCA) and centrifuged at 3000 g for 3 min. 75 μl supernatant or standard was added to 2775 μl of phosphate/EDTA buffer (1 litre containing 13.6 g KH₂PO₄, 1.86 g EDTA, pH 8) followed by 150 μl of 0.1% o-phthaldehyde (OPT) in methanol. The samples were gently mixed and kept at room temperature for 30 min. Absorbance was read on a fluorimeter at excitation wavelength 350 nm and emission wavelength 420 nm.

The glutathione content after exposure to GalN are shown in FIG. 6. Glutathione content at 20 mM and lower concentrations of GalN did not decrease but at 40 mM of GalN, glutathione was significantly decreased (p<0.05).

5. Lactate Dehydrogenase (LDH) Leakage

LDH in the media was assayed using the method developed by Korzeniewski and Callewaert (Korzeniwski, C., and Callewaert, D. M. (1983). An enzyme-release assay for natural cytotoxicity. J Immun. Meth. 64, 313-320). Samples of 100 μl of either spheroid homogenate or medium were added to 96-well plates along with 100 μl LDH reagent containing 54 mM L(+) lactate, 1.3 mM β-NAD, 0.28 mM phenazine methosulphate, 0.66 mM p-iodonitrotetrazolium violet in 0.2 M Tris buffer, pH 8.2). The resultant absorbance at 490 mn was monitored for 15 min, at 10 sec intervals on a microplate reader (Multiscan RC, Labsystem).

LDH leakage of liver spheroids 24 hr after GalN exposure are shown in FIG. 3. Compared with the control, the significant increase of LDH leakage at 20 and 40 mM of GalN was detectable 4 hours after exposure (p<0.05). At 24 hr, LDH leakage at 10 mM. of GalN was also significantly increased (p<0.05). LDH leakage showed a concentration-dependent increase.

### Conclusions

This study has shown that GalN at 20 mM caused a functional change but did not decrease protein and glutathione levels. Whereas GalN at 40 mM significantly decreased protein and glutathione levels in addition to inducing other toxic changes. Significant protein loss reflects a large percentage of cell death. The decrease in glutathione at the highest concentration may contribute to GalN hepatotoxicity, however, it is not clear whether the reduction in glutathione is induced by GalN or is a consequent of cell death. Using the liver spheroid test, it can be concluded that glutathione is unlikely to be involved in the initiation of GalN-induced hepatotoxicity because GalN at 20 mM caused functional changes but did not affect glutathione levels. So selecting a suitable concentration range is crucial for in vitro studies, especially for biochemical evaluations after intoxication or it may lead to an over- or under-estimate of toxicity.

1. A method for the formation of spheroids comprising:
(a) obtaining and/or deriving a plurality of cells from a tissue or an organ or from a part thereof;
(b) orbitally rotating the cells in a fluid medium at a first speed and for a time sufficient for spheroids to form; and
(c) further orbitally rotating the spheroids formed in step (b) in a fluid medium at a second speed or speeds, slower than said first speed.

2. A method according to claim 1, wherein the cells in step (c) are rotated at a second speed of less than 80 rpm.

3. A method according to claim 1 or claim 2 wherein the cells in step (b) are rotated at a first speed of at least 77 rpm.

4. A method according to claim 3 wherein the cells in step (b) are rotated at a first speed of from 77 to 90 rpm.

5. A method according to claim 4 wherein the cells in step (b) are rotated at a first speed of from 83 to 87 rpm.

6. A method according to any preceding claim wherein the duration of rotation of the cells in step (a) is from 1 to 4 days.

7. A method according to claim 6 wherein the duration of rotation of the cells in step (a) is from 1 to 2 days.

8. A method according to any preceding claim wherein the duration of rotation of the cells in step (b) is from 2 to 45 days.

9. A method according to claim 8 wherein the duration of rotation of the cells in step (b) is from 2 to 35 days.

10. A method according to any preceding claim further comprising a step (d) of cryopreserving the spheroids produced in step (c).

11. A method according to any preceding claim wherein the cells of step (a) are derived from a mammalian or avian tissue or organ or cell-line.

12. A method according to claim 11 wherein the mammalian tissue or organ or cell-line is human, non-human, primate, porcine or rodent.

13. A method according to claim 11 wherein the avian tissue is chick tissue.

14. A method according to any preceding claim which is carried out such that in the resulting spheroid population at least 75% of the spheroids have a diameter of from 100 μm to 350 μm.

15. A method according to claim 14 wherein at least 75% of the spheroids have a diameter of from 150 μm to 200 μm.

16. A method according to claim 15 wherein at least 90% of the spheroids have a diameter of from 100 μm to 350 μm.

17. A method according to any one of claims 1 to 16, wherein at least 90% of the spheroids have a diameter of from 150 μm to 200 μm.

18. A method according to any preceding claim, wherein each spheroid consists of a reaggregate of at least 10³ cells.

19. A method according to any preceding claim, wherein the spheroids are morphologically regular in shape.

20. A method according to any preceding claim wherein the spheroids have a smooth surface.

21. A spheroid produced by a method according to any preceding claim.

22. An in vitro model comprising a spheroid produced by a method according to any one of claims 1 to 20.
